(19) United States
(12) Patent Application Publication
(10) Pub. No.: US 2022/0233221 A1
(43) Pub. Date: Jul. 28, 2022

(54) BALANCED AXIAL COMPRESSION ASSEMBLY FOR BONE

(71) Applicant: DBKAY DESIGNS, LLC, AKRON, OH (US)

(72) Inventors: David B. KAY, Akron, OH (US); Ian KAY, Fairlawn, OH (US); Andrew Leither, Akron, OH (US)

(21) Appl. No.: 17/614,918

(22) PCT Filed: Jun. 11, 2020
(86) PCT No.: PCT/US2020/037137

§ 371 Date: Nov. 29, 2021

Related U.S. Application Data
(60) Provisional application No. 62/861,692, filed on Jun. 14, 2019.

(51) Int. Cl. A61B 17/80; A61B 17/86

(52) U.S. Cl. CPC A61B 17/8014; A61B 17/8605; A61B 17/864

(57) ABSTRACT

There is disclosed devices and methods for providing continuous bone compression for bone healing which includes a bone plate that is secured to bone segments to be fused by a first cannulated bone screw and a second cannulated bone screw. A first elastic loop is fixed to the first and second bone screw in a first plane and a second elastic element is fixed to the first and second bone screws in one or more second planes.

### BACKGROUND OF THE INVENTION

The invention relates generally to orthopedic medical devices and methods, and more specifically, to orthopedic implant devices and methods for applying balanced axial compression for bone healing.

In the past, surgical optimization for bone healing was often predicated on bone being a static material. However, bone is living, dynamic system and constantly changes based on a variety of biologic and mechanical factors. For example, bone resorption typically occurs at the interface of two bone surfaces. In particular, this is the case, when the bone is incorporated into an orthopedic construct that includes the bone, fixation members, such as screws, and a scaffold member, such as a plate. This bone resorption changes the construct mechanics, for example, at the screw location such that a less than optimal construct evolves over time. This can lead to failure of bone fusion or healing. Conventional means to address this problem is to rely upon advanced elastic materials, such as nitinol and nitinol polymer compounds. While these materials can be structured to provide continuous compression, they present certain disadvantages. For example, they are often brittle, and difficult to shape. They are expensive, and they are not “tunable”, meaning that they are not able to achieve a direction of an amount of force that can be tailored to a specific quality of bone, which results in either insufficient compression or so much compression that the implants can damage the bone, leading to bone necrosis, inflammation and failure of the bone to unite.

### SUMMARY OF THE INVENTION

The invention is directed to a orthopedic plate and screw assembly that is configured to reside on the surface of a bone, including a) at least one plate having at least one aperture therein; and (b) at least one elastomeric and/or metal member in operative engagement with the plate and screw assembly and configured to apply a compressive force directly or indirectly to one or more bones or separate pieces of a bone. As used herein, “plate” refers to a device that generally has a first surface with a second spaced surface that at least for a portion, generally corresponds to the topography of the first surface such that the through thickness is the same at more than one location of the “plate.” The plate may include a radius in one or more directions, for example across the width of the bottom surface of the plate.

In this embodiment, the plate assembly has a longitudinal axis and includes a first plate member having an internal recess within a surface, such as the top surface of the first plate member. The plate member has at least two apertures to receive fixation members, such as bone screws, pins, or nails that extend downward, and nominally normal to the medial plane of the plate. Preferably, at least one of the fixation member, i.e. a screw, is a locking fixation member which is fixed in its orientation relative to the plate and/or plate aperture that holds it, and a second fixation member resides in an elongated slot which enables the screw to move toward the locking screw when a compressive force is applied to it. The plate assembly further includes a first elastic element which elastically couples the first and second fixation members in one plane and a second elastic element that couples the first and second fixation members.

The elastic element can be an elastic ring, an elastic cable, and retractable ring members that may have spring qualities. It is preferable that the elastic element applies the compressive force in a balanced manner on two sides of the longitudinal axis of the plate assembly. The first elastic element can encircle the peripheries of the heads of the first and second fixation members, or can reside between a groove in the heads of the fixation members and the internal side edges of a recess in the top surface of the plate. The second elastic element extends down a cannulation in the first fixation member, across a gap between the first and second fixation member and up a cannulation in the second fixation member.

In a still further embodiment, the invention is directed to a method of providing balanced multi-planar bone compression for bone healing including the step of providing a plate member onto the surface of a bone segment by fixing fixation members through a first fixation member aperture and a second fixation member aperture, at least one of which is slotted, and engaging the first and second fixation members with an elastic ring to shorten a distance between the first and second fixation members along the longitudinal axis of the plate so as to apply a compressive force between fixation members which extend into bone. Subsequently, a second elastic member is threaded through a cannulation in the first fixation member across a gap between the first and second fixation member and up a cannulation in the second fixation member. The second elastic element is secured in the first fixation member and in the second fixation member at an opposite end.

### BRIEF DESCRIPTION OF THE DRAWING FIGURES

FIG. 1 is a top perspective view of a bone compression assembly in accordance with an aspect of the invention;

FIG. 2 is a top view of the device of FIG. 1;

FIG. 3 is a detail of the torque receiving recess of the device shown in FIG.4;

FIG. 4 is a side view of a fixation member for use in the present invention;

FIG. 5 is a side cross section taken through line O-O of FIG. 4;

FIG. 6 is a side view of the assembly of FIG. 1;

FIG. 7 is an exploded top side perspective view of the bone compression assembly of FIG. 1;

FIG. 8 is across sectional view of the device of FIG. 7 taken along the medial longitudinal axis;

FIG. 9 is a top side perspective view of the bone compression device of FIG. 1 illustrating a step of inserting the second elastic element in a cannulation in a first fixation member;

FIG. 10 is a view of the device of FIG. 9 showing a step of retrieving the second elastic member through the second fixation member cannulation;

FIG. 11 illustrates the step of tensioning the second elastic element in the second fixation member; and

FIG. 12 illustrates a cross section of the bone compression assembly of FIG. 11.

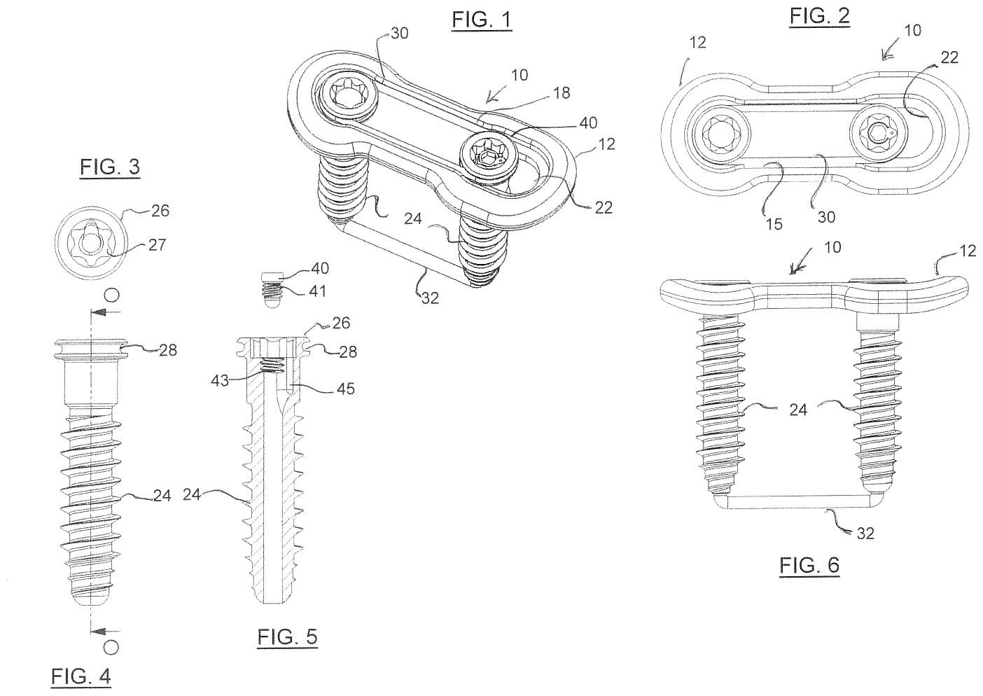

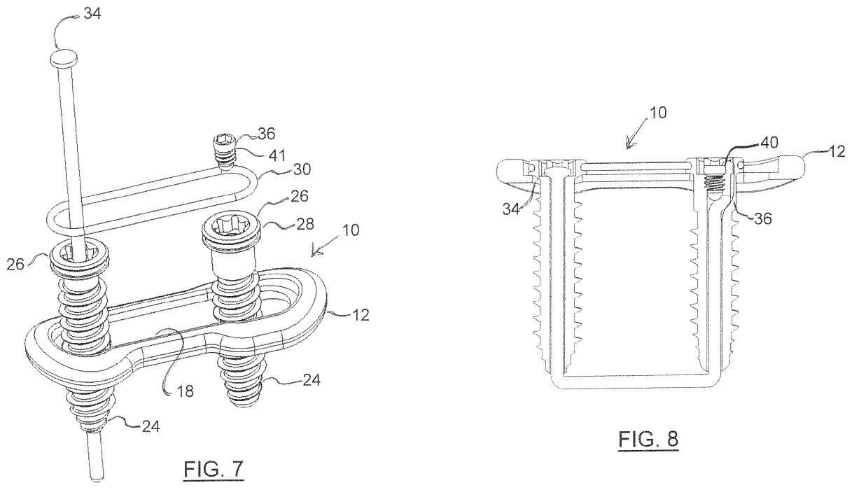

### DETAILED DESCRIPTION OF THE INVENTION

The invention presents multiple “tunable” methods that will allow for directed scalable continuous constant, or variable compression of bone segments to facilitate for bone healing in fracture repair, bone osteotomies and bone fusions. Similarly, these devices can be used in soft tissue repair to address the inherent creep that occurs to ligaments and tendons that are used in reconstruction.

The invention provides for the application and balancing of forces that are used to apply a compressive force to associated bone. In particular, the bone compression device or assembly 10 of the present invention provides a bone plate 12 having at least two apertures 14 that receive fixation members 16, such as bone screws. The plate further includes a recess 18 surrounding the apertures. Preferably one of the aperturesl4 is a round screw hole 20 and the second aperture is a slotted aperture 22. The screw 24 that is received in the slotted aperture includes a head 26 having an external peripheral groove 28, as may the locking screw member 25. The screw head 26 further has a torque driving recess 27. A looped elastic element 30 is captured between the side walls of the recess and in the groove around the head or heads of the bone screws.

The bone screws are cannulated along their long axis, and a second elastic element 32, such as an elastic cable is passed through the first screw cannulation, across a gap between the screws, and the second elastic element is retrieved up the cannulation of the second fixation member, and it is tethered in the first screw by means of an enlarged stop member 34, and is held in the second screw member by a set screw 36. The second screw member has a compound cannulation so that the set screw 36 can be fixed in internal threads 43 by means of external threads 41 on the set screw 36. The cannulation also includes a recess 45 to one side. The band is tethered within the recess 45 by means of a rim 40 on the set screw which wedge the band into the recess 45.

The ideal conditions for bone healing are well documented and include compression that allows for minimal micromotion; reduction in torsion at the bone surface interface for fracture healing, reconstruction osteotomies and fusions; minimal surgical disruption to the blood supply so as to avoid or reduce disvascular bone and cause further inflammation to the healing site; and fixation that is overly rigid and creates stress shielding and prevents bone healing.

The invention describes both endoskeletal and exoskeletal methods to create continuous compression in bone that is physiologically axially loaded and bone that is not axially loaded in use. Physiological axial bone loading is seen in the femur and the tibia during standing, for example. The axial loading, that occurs with weight bearing, is leveraged using intramedullary rod fixation that has a fixed end and the opposite site of fixation is allowed to slide in one plane. However, if there is no weight bearing the loading does not occur. In addition, the loading to the bone surface varies based upon the individual patient's abilities to load the bone. Physiologically non-axially loaded bones, such as in the midfoot, tend to undergo shear with weight bearing and may be a causative factor in the reported high incidence of non-healing bone.

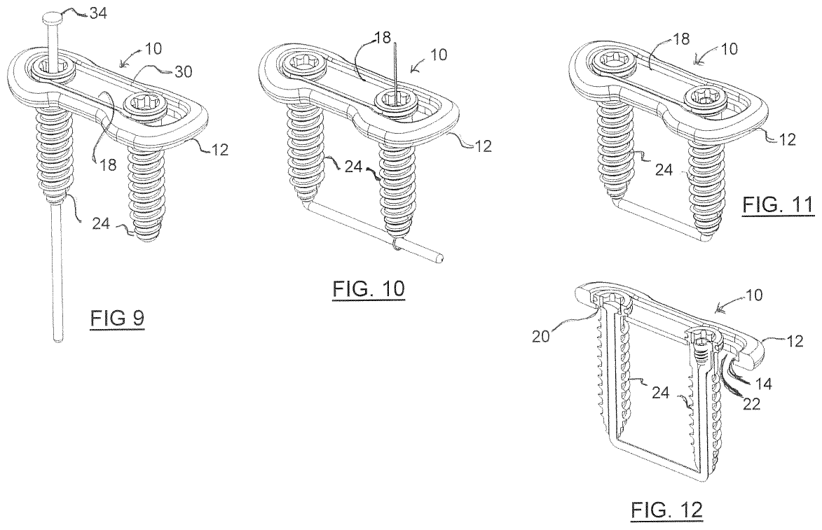

FIGS. 9-11 illustrate a method of fusing bone by applying compression through the implantation of the bone compression assembly of the present invention. First the bone is prepped and the plate is placed on the top surface of the bone segments. The first and second bone screws are inserted through apertures in the plate and a first elastic loop is feed around and into grooves in the heads of the first and second bone screws. Then a second elastic cable is feed in a first bone screw through a cannulation. A suture passer is feed through a cannulation in the second bone screw and retrieved upward through that cannulation. A set screw is inserted into a threaded passage in the head of the second bone screw to fix the second elastic cable under tension down through the first bone screw across the gap between the bone screws, and up through the cannulation in the second screw.

While certain representative embodiments and details have been shown for the purpose of illustrating the subject invention, it will be apparent to those skilled in this art that various changes and modifications can be made therein without departing from the scope of the subject invention. In this regard, the scope of the invention is to be limited only by the following claims.

What is claimed is:

1. An orthopedic implant for a bone or bone segment comprising:
a construct defining a construct axis and having a first aperture with at least one first fixation member having a proximal portion having a neck and a head and a long axis capable of fixing the first fixation member to the bone segment, and a second slotted aperture member with a second fixation member having a proximal portion having a neck and a head and a long axis and capable of fixing the second fixation member to the bone segment; and
a first elastic loop compression mechanism operatively coupled to a proximal portion of the first fixation member and to a proximal portion of the second fixation member and capable of applying a force to the first fixation member and the second fixation member in the direction of the construct axis and a second elastic element operatively coupled to the first fixation member and to the second fixation member along the long axis of the first fixation member and the long axis of the second fixation member.

2. An orthopedic implant as set forth in claim 1, wherein the fixation member is a screw, nail or pin.

3. An orthopedic implant as set forth in claim 1, wherein the first fixation member is a cannulated screw and the second fixation member is a cannulated screw.

4. An orthopedic implant as set forth in claim 1, wherein the first and the second fixation member each has a head which includes a peripheral groove that captures the first elastic loop compression mechanism

5. An orthopedic implant as set forth in claim 1, wherein the construct is an orthopedic plate.

6. An orthopedic implant as set forth in claim 5, wherein the plate has a recess that houses the head of the first fixation member and the second fixation member.

7. An orthopedic implant as set forth in claim 1, wherein the second elastic element is an elastomeric band.

8. An orthopedic implant as set forth in claim 7, wherein the first fixation member and the second fixation member each include a cannulation along the respective long axis and the elastomeric band is received in the cannulation of each of the first fixation member and the second fixation member.

9. An orthopedic implant as set forth in claim 8, wherein at least one of the first fixation member and the second fixation member include a set screw that is received in a respective cannulation to secure the band relative to the fixation member.

10. A method for the fixation of bone comprising the steps of:
preparing a surface between two bones for fusion;
placing a bone plate on a second surface of each of the two bones,
securing a first fixation member to one of the two bone by inserting it through an aperture in the bone plate,
securing a second fixation member to the other of the two bones by inserting it through an aperture in the bone plate,
looping an elastic element around the first fixation member and the second fixation member in a first plane, and
feeding a second elastic element through a first cannulation in the first fixation member and through a second cannulation in the second fixation member.

11. A method for the fixation of bone as set forth in claim 10, including the further step of fixing the second elastic element in the first fixation member.

12. A method for the fixation of bone as set forth in claim 11, wherein the cannulation has a diameter and the second elastic element includes a stop which has a diameter which is greater than the diameter of the first cannulation.

13. A method for the fixation of bone as set forth in claim 11, including the further step of fixing the second elastic element in the second fixation member.

14. An orthopedic implant for a bone or bone segment comprising:
an orthopedic plate defining a plate axis and having a first aperture with at least one first fixation member having a first cannulation and which is a screw, nail, staple or pin and having a proximal portion having a neck and a head and a long axis capable of fixing the first fixation member to the bone segment, and a second slotted aperture member with a second fixation member having a second cannulation and a proximal portion having a neck and a head and a long axis and capable of fixing the second fixation member to the bone segment; and
a first elastic loop compression mechanism which engages the neck or the head of the first fixation member and the neck or the head of the second fixation member and capable of applying a force to the first member and the second member in the direction of the plate axis and a second elastic element which is received in the first cannulation and in the second cannulation.

15. An orthopedic implant as set forth in claim 14, wherein the first fixation member is a cannulated screw and the second fixation member is a cannulated screw which includes a set screw that secures the second elastic element in the second cannulation.

16. An orthopedic implant as set forth in claim 14, wherein the first elastic loop compression mechanism is an elastomeric loop.

17. An orthopedic implant as set forth in claim 14, wherein the first and the second fixation member each has a head which includes a peripheral groove that captures the first elastic loop compression mechanism.

18. An orthopedic implant as set forth in claim 16, wherein the plate has a recess that houses the head of the first fixation member and the second fixation member and the elastomeric loop.

19. An orthopedic implant as set forth in claim 18, wherein the second elastic element is an elastomeric band.

20. An orthopedic implant as set forth in claim 19 ,wherein the first cannulation has a diameter and the second elastic element includes a first end having a stop which has a diameter that is greater than the diameter of the first cannulation.
